(19) United States
(12) Patent Application Publication
(10) Pub. No.: US 2022/0125702 A1
(43) Pub. Date: Apr. 28, 2022

(54) COMPOSITION COMPRISING A FATTY SUBSTANCE, A PEG POLYMER, A DIOL AND A POLYSACCHARIDE

(71) Applicant: L'OREAL, Paris (FR)

(72) Inventors: Gwenaëlle JEGOU, AX WASSENAAR (NL); Sylvia DA SILVA, SAINT-OUEN (FR); Audrey GILLES, SAINT-OUEN (FR); Ségolène DE MENTHIERE, SAINT-OUEN (FR)

(21) Appl. No.: 17/312,785

(22) PCT Filed: Dec. 16, 2019
(86) PCT No.: PCT/EP2019/085376

§ 371 Date: Jun. 10, 2021

(30) Foreign Application Priority Data
Dec. 21, 2018 (FR) 1873866

(51) Int. Cl. A61K 8/86; A61K 8/34; A61K 8/37; A61K 8/73; A61K 8/9794; A61K 8/41; A61Q 5/06; A61K 8/898

(52) U.S. Cl. CPC A61K 8/86; A61K 8/345; A61K 8/342; A61K 8/37; A61K 8/898; A61K 8/9794; A61K 8/416; A61Q 5/06; A61K 8/73

(57) ABSTRACT

The present invention relates to a composition, in particular a cosmetic hair composition, comprising one or more fatty substances, one or more polysaccharides in an amount of at least 3.0% by weight of the total weight of the composition, one or more diols having a molecular weight of less than 1000, and one or more polyethylene glycols comprising from 10 000 to 200 000 oxyethylene units. The invention also relates to a process for treating keratin fibers, in particular the hair, with the composition of the invention. This invention allows in particular the shaping and/or conditioning of keratin fibers, and in particular for defining the curls of the hair and fixing the shaping of dry hair.

### CROSS REFERENCE TO RELATED APPLICATION

This is a national stage application of PCT/EP2019/085376, filed internationally on Dec. 16, 2019, which claims priority to French Application No. 1873866, filed on Dec. 21, 2018, which is incorporated by reference herein in its entirety.

The present invention relates to a composition, in particular a cosmetic composition, in particular a hair composition, comprising one or more fatty substances, one or more polyethylene glycols, one or more particular diols and one or more polysaccharides.

The invention also relates to a process for shaping and/or conditioning keratin fibers, in particular human keratin fibers such as the hair, comprising at least one step of applying to said fibers the composition according to the invention.

Lastly, the invention relates to the use of the composition according to the invention for shaping and/or conditioning keratin fibers, in particular human keratin fibers such as the hair, and in particular for defining the hair curls or styling the head of hair.

Many individuals are unsatisfied with the appearance of their hair: in particular people who have curly hair may wish to maintain the curls but to obtain better curl definition and better regularity, better movement and less volume and frizziness. Furthermore, some individuals find it difficult to control, define and/or style their curly hair. People are also searching for the possibility of styling or giving volume to or shaping the head of hair.

There is a need to provide compositions which make it possible to simplify the styling routine of individuals who wish to temporarily change their hair. There is a need to provide a unique composition that allows application to wet hair or dry hair with a particular galenic presentation which is different as required and which may vary depending on the desired result.

In particular, there is a need to provide compositions which are stable over time and which make it possible to style wet hair and to give the curls good definition when this hair is curly, and fix the hairstyle on dry hair while at the same time giving the the hair a good cosmetic feel.

Thus, a subject of the present invention is a composition for treating keratin fibers, in particular human keratin fibers such as the hair, comprising the combination of one or more fatty substances, one or more polyethylene glycols, one or more diols having a molecular weight of less than 1000, and one or more polysaccharides in a total content of greater than or equal to 3% by weight relative to the total weight of the composition.

This composition has a particular galenic form that allows the application of a liquid composition to wet hair to provide a styling effect or a curl definition but also allows a finishing application, to dry hair, of a composition in which is thready after said composition has been worked in the hands. This second galenic form makes it possible not to “wet” the hair, which would result in at least a partial loss of the shape that the user wishes to fix/preserve. It is important for this particular galenic form to persist after storage.

For the purposes of the invention, the term “storage stable” is intended to mean more particularly that the properties of formation of a thready composition persist after storage at 45° C., in particular after 5 weeks of storage.

In addition, the composition according to the invention affords a hair conditioning effect and makes it possible in particular to give a pleasant cosmetic feel, notably a smooth feel, to facilitate the disentangling of the hair and to provide sheen.

A subject of the invention is also a process for shaping and/or fixing keratin fibers, in particular human keratin fibers such as the hair, comprising at least one step of applying to said fibers the composition according to the invention.

Other subjects, characteristics, aspects and advantages of the invention will emerge even more clearly on reading the description and the example that follows.

In the present description, the expression “at least one” is equivalent to the expression “one or more” and can be substituted for said expression; the expression “between” is equivalent to the expression “ranging from” and can be substituted for said expression, and implies that the limits are included.

### DETAILED DESCRIPTION

### Fatty Substance

The term “fatty substance” is intended to mean an organic compound that is insoluble in water at ordinary ambient temperature (25° C.) and at atmospheric pressure (760 mmHg) (solubility of less than 5%, preferably less than 1% and even more preferentially less than 0.1%). They have in their structure at least one hydrocarbon-based chain comprising at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

These fatty substances are neither polyoxyethylenated nor polyglycerolated. They are different from fatty acids since salified fatty acids constitute soaps which are generally soluble in aqueous media.

The fatty substances are in particular chosen from C₆-C₁₆ hydrocarbons or hydrocarbons comprising more than 16 carbon atoms and in particular alkanes, oils of animal origin, oils of plant origin, glycerides or fluoro oils of synthetic origin, fatty alcohols, fatty acid and/or fatty alcohol esters, non-silicone waxes, and silicones.

It is recalled that, for the purposes of the invention, the fatty alcohols, fatty esters and fatty acids more particularly contain one or more linear or branched, saturated or unsaturated hydrocarbon-based groups comprising 6 to 30 carbon atoms, which are optionally substituted, in particular with one or more (in particular 1 to 4) hydroxyl groups. If they are unsaturated, these compounds may comprise one to three conjugated or unconjugated carbon-carbon double bonds.

As regards the C₆-C₁₆ alkanes, they are linear or branched, and possibly cyclic. Examples that may be mentioned include hexane, dodecane and isoparaffins such as isohexadecane and isodecane. The linear or branched hydrocarbons containing more than 16 carbon atoms may be chosen from liquid paraffins, petroleum jelly, liquid petroleum jelly, polydecenes, and hydrogenated polyisobutene such as Parleam©.

According to a particular embodiment, the fatty substance(s) used in the process of the invention are chosen from fatty alcohols and/or fatty esters.

The fatty alcohols that may be used in the cosmetic compositions of the invention are saturated or unsaturated, and linear or branched, and comprise from 6 to 30 carbon atoms and more particularly from 8 to 30 carbon atoms. Examples that may be mentioned include cetyl alcohol, stearyl alcohol and the mixture thereof (cetylstearyl alcohol), octyldodecanol, 2-butyloctanol, 2-hexyldecanol, 2-undecylpentadecanol, oleyl alcohol and linoleyl alcohol.

The wax(es) that may be used in the cosmetic composition of the invention are chosen especially from carnauba wax, candelilla wax, esparto grass wax, paraffin wax, ozokerite, plant waxes, for instance olive tree wax, rice wax, hydrogenated jojoba wax or the absolute waxes of flowers such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and polyethylene waxes or polyolefin waxes in general.

As regards the fatty acid and/or fatty alcohol esters, which are advantageously different from the triglycerides mentioned above, mention may be made in particular of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters more particularly being greater than or equal to 10.

Among the monoesters, mention may be made of dihydroabietyl behenate;

octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononate; isostearyl palmitate; methyl acetyl ricinoleate; myristyl stearate; octyl isononanoate; 2-ethylhexyl isononanoate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as fatty ester, sugar esters and diesters of C₆-C₃₀ and preferably C₁₂-C₂₂ fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be notably chosen from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates and arachidonates, or mixtures thereof such as, in particular, oleopalmitate, oleostearate and palmitostearate mixed esters.

More particularly, use is made of monoesters and diesters and especially sucrose, glucose or methylglucose monooleate or dioleate, stearate, behenate, oleopalmitate, linoleate, linolenate or oleostearate.

An example that may be mentioned is the product sold under the name Glucate® DO by the company Amerchol, which is a methylglucose dioleate.

Examples of esters or mixtures of esters of sugar and of fatty acid that may also be mentioned include:
the products sold under the names F160, F140, F110, F90, F70 and SL40 by the company Crodesta, respectively denoting sucrose palmitate/stearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referenced B370 and corresponding to sucrose behenate formed from 20% monoester and 80% diester-triester-polyester;
the sucrose mono-dipalmito-stearate sold by the company Goldschmidt under the name Tegosoft® PSE.

According to one variant of the composition of the invention, at least one of the fatty substances is solid. Preferably, among all the fatty substances, the solid fatty substance(s) are present in a major total amount compared to all the fatty substances present in the composition. According to another embodiment, all the fatty substances of the composition are solid.

The term “solid fatty substance” is intended to mean a fatty substance that is solid at 25° C. and at ambient temperature.

According to one particular embodiment, the fatty substance is chosen from C₁₂-C₂₀, preferably C₁₆-C₁₈, fatty alcohols, such as cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol, preferably cetyl and/or stearyl alcohol, preferably cetylstearyl alcohol. The fatty esters are chosen from esters of a fatty acid and of myristyl alcohol, such as myristyl stearate or myristyl palmitate, alone or as a mixture.

According to one particular embodiment, the composition of the invention comprises a mixture of fatty alcohols and fatty esters obtained from a fatty alcohol and a fatty acid.

According to one preferred embodiment, the composition according to the invention comprises one or more solid fatty alcohols, and one or more solid fatty esters, preferably obtained from fatty alcohol(s) and fatty acid(s).

Preferably, the composition of the invention comprises, as a mixture, stearyl alcohol, cetyl alcohol, myristyl stearate and myristyl palmitate.

The composition according to the invention may comprise a total amount of fatty substances ranging from 0.01 to 20% by weight, preferably from 0.1 to 10% by weight, more preferentially from 1 to 8% by weight and better still from 2 to 5% by weight, relative to the total weight of the composition.

The composition according to the invention may comprise a total amount of solid fatty substances ranging from 0.01 to 20% by weight, preferably from 0.1 to 10% by weight, more preferentially from 1 to 8% by weight and better still from 2 to 5% by weight, relative to the total weight of the composition.

### Polyethylene Glycol

The composition of the invention comprises one or more high molecular-weight polyethylene glycols. The polyethylene glycols (PEGs) that are of use in the invention have the following structure (A),

H—(O—CH₂CH₂)ₙ—OH

(A)

wherein n may vary from 10 000 to 200 000, preferably from 50 000 to 100 000.

By way of example, mention may be made in particular of the polyethylene glycols produced by Dow under the INCI name PEG-2M, PEG-5M, PEG-7M, PEG-14M, PEG-23M, PEG-45M, PEG-90M, PEG-160M, and PEG-180M known as Polyox™. According to one particular embodiment, the polyethylene glycol is PEG-90M.

According to one variant, the polyethylene glycol comprises an average number of oxyethylenated units of between 80 000 and 100 000.

According to the present invention, the polyethylene glycol(s) can be present in a total amount of between 0.01% and 20% by weight, preferably from 0.1% to 10% by weight, preferentially from 0.2% to 5% by weight, better still from 0.3% to 1% by weight of the total weight of the composition.

### Polysaccharides

As indicated above, the composition according to the invention comprises one or more polysaccharides in a total amount of greater than or equal to 3.0% by weight relative to the total weight of the composition.

Preferably, the polysaccharide(s) according to the invention are chosen from nonionic, anionic and amphoteric polysaccharides.

For the purposes of the present invention, the term “polysaccharides” refers to a polymer comprising sugar units. The term “sugar unit” refers to an oxygen-bearing hydrocarbon-based compound containing several alcohol functions, with or without aldehyde or ketone functions, and which includes at least 4 carbon atoms.

The sugar units may be optionally modified by substitution, and/or by oxidation and/or by dehydration.

The sugar units that may be included in the composition of the polysaccharides of the invention are generally derived from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

Polysaccharides that may notably be mentioned include native gums such as

a) tree or shrub exudates, including:
gum arabic (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (polymer of galacturonic acid, galactose, fucose, xylose and arabinose);

b) gums derived from algae, including:
agar (polymer derived from galactose and anhydrogalactose);
alginates (polymers of mannuronic acid and of glucuronic acid);
carrageenans and furcellerans (polymers of galactose sulfate and of anhydrogalactose sulfate);

c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose);

d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);
pullulan gum;

e) polymers extracted from plants, including:
celluloses (glucose polymers);
starches (glucose polymers) and
inulin.

By way of polysaccharides according to the invention, mention may in particular be made of anionic or nonionic microbial gums.

For the purposes of the present invention, “microbial gums” is intended to mean substances synthesized by fermentation of sugars by microorganisms.

The microbial gums can be chosen from scleroglucan gums, gellan gums, pullulan gums, curdlan gums, xanthan gums, grifolan gums, lentinan gums, schizophyllan gums, spirulinan gums and krestin gums.

Mention may in particular be made of the scleroglucan gums produced by Sclerotium rolfsii, the gellan gums produced by Pseudomonas elodea or Sphingomonias, the pullulan gums produced by Aureobacidium pullulens, the curdlan gums produced by Alcaligenes of Faecalis myxogènes type, the xanthan gums produced by numerous organisms, including Leuconostoc mesenteroides and Leuconostoc dextrantum, the grifolan gums produced by Grifola frondara, the lentinan gums produced by Lentinus edodes, the schizophyllan gums produced by Schizophyllum commine, the spirulinan gums produced by Spirulina sybsyla and the krestin gums produced by Coriates versicolor.

The polysaccharide(s) of the composition according to the invention may also be mixtures of polysaccharides chosen from the above polysaccharides.

Preferably, the polysaccharide(s) according to the invention are preferably chosen from microbial gums. Preferably, the polysaccharide(s) according to the invention is(are) chosen from scleroglucan gum and/or pullulan gum.

The scleroglucans used in accordance with the invention are neutral polysaccharides preferably corresponding to formula (I):

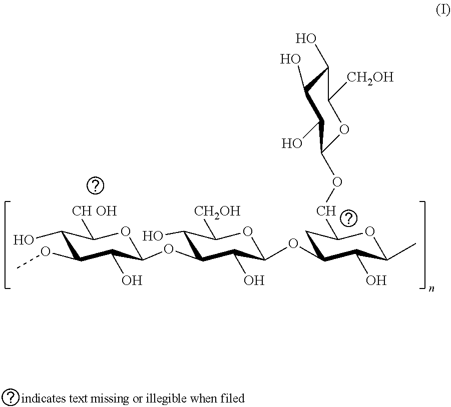

where the degree of polymerization n ranges from 500 to 1600.

According to the invention, use is advantageously made of scleroglucans of microbial origin, obtained for example by aerobic fermentation of a glucose-containing medium by a fungus of the Sclerotium type having the structure of a D-glucopyranose homopolymer.

Examples of scleroglucan gums that may be used in the present invention are, in a non-limiting manner, the products sold under the name Actigum CS, in particular Actigum CS 11 by the company Sanofi Bio Industries and under the name Amigum by the company Alban Müller International.

Other scleroglucans, such as that treated with glyoxal described in French patent application No. 2,633,940, may also be used.

According to one particular embodiment, the polysaccharide(s) are chosen from scleroglucan gums and pullulans, alone or as a mixture, preferably as a mixture.

According to one variant, the total content of the polysaccharide(s) is generally between 3.0% and 20% by weight, preferentially from more than 3.5% to 10% by weight, and even better still between 4% and 7% by weight relative to the total weight of the composition.

### Diols

The composition according to the invention comprises at least one diol having a a molecular weight of less than 1000.

Preferably, the diol(s) according to the invention are chosen from diols having a molecular weight of less than 500, preferentially less than 100.

By way of example of diols having a molecular weight of less than 1000, mention may be made of propylene glycol (or 1,2-propanediol), dipropylene glycol, 1,3-propanediol, pinacol (2,3-dimethyl-2,3-butanediol), 2,3-butanediol, 1,3-butanediol, octane-1-2 diol, 2-methyl-1,3-propanediol and mixtures thereof. Preferably, the diol is butylene glycol.

According to one variant, the composition according to the invention comprises one or more diols having a molecular weight of less than 1000 in a total amount ranging from 0.1 to 25% by weight, preferentially from 0.5 to 20% by weight, better still 1 to 15% by weight, even better still from 3 to 10% by weight, relative to the total weight of the composition.

According to the present invention, the composition is provided as a styling milk and preferably has a viscosity of less than 5000 mPa·s, preferentially less than 3000 mPa·s.

The viscosity can be measured using a rheometer such as Rheomat 180 (Mettler) equipped with a spindle 3 (200 revolutions/min, 25° C., measured at 30 s).

According to one particular embodiment, this composition is used in a pump dispenser bottle which makes it possible to obtain in the hand a pre-metered amount of product in milk form. When the composition leaves the dispenser bottle in milk form, it is generally applied to wet hair for direct application of the composition to the head of hair.

When it is desired to apply this composition to shaped dry hair, it is preferable to massage this liquid composition between the hands to obtain a texture formed of texturizing threads or fibers when the palms of the hands are separated and then to apply these texturizing fibers to dry hair in order to fix the shape of the hair.

The composition according to the invention may also comprise one or more additional compounds chosen from fixing polymers, surfactants, preferably chosen from nonionic, anionic, cationic and amphoteric surfactants, natural or synthetic thickeners or viscosity regulators other than the compounds described above; UV filters, fillers such as nacres, titanium dioxide, resins and clays, fragrances, peptizers, vitamins, preserving agents, acidic agents, alkaline agents.

Those skilled in the art will take care to choose any additional compounds and the amount thereof so that they do not adversely affect the properties of the composition of the present invention, in particular in terms of fibrous texture after massaging and of stability of this effect over time.

These additives may be present in the composition according to the invention in an amount ranging from 0% to 20% by weight relative to the total weight of the composition.

The composition according to the invention is preferably aqueous. The water content is preferentially greater than or equal to 50% by weight, better still ranges from 60 to 95% by weight, even better still ranges from 70 to 90% by weight, relative to the total weight of the composition.

The pH of the composition according to the invention generally ranges from 2 to 12, preferably from 2.5 to 7, preferentially from 2.5 to 6 and better still from 2.5 to 4.

The pH of these composition may be adjusted to the desired value by means of basifying agents or acidifying agents that are customarily used. Among the basifying agents, examples that may be mentioned include aqueous ammonia, alkanolamines, and mineral or organic hydroxides. Among the acidifying agents, examples that may be mentioned include mineral or organic acids, for instance hydrochloric acid, orthophosphoric acid or sulfuric acid, carboxylic acids, for instance acetic acid, tartaric acid, citric acid and lactic acid, and sulfonic acids.

Another subject of the present invention is a process for shaping and/or conditioning keratin fibers, in particular human keratin fibers such as the hair, comprising at least one step of applying to said fibers a composition as described previously.

According to one particular embodiment of the process, the composition is applied directly to wet or dry hair, preferably wet hair, in the liquid state.

According to a second embodiment, the liquid composition is massaged in the palms of the hands to obtain a thready texture which is then applied to wet or dry hair, preferably dry hair.

This or these steps of applying the composition according to the invention may optionally be preceded by at least one step of washing said fibers using a shampoo.

Moreover, this or these steps of applying the composition according to the invention may optionally be followed by a drying step when this composition is applied to wet hair.

A subject of the present invention is also a use of the composition as described above, for shaping and/or conditioning keratin fibers.

Preferably, the composition as described above is used on wet and curly hair, for a better curl definition or on dry hair for the maintaining the hairstyle.

The examples that follow serve to illustrate the invention without, however, being limiting in nature.

### EXAMPLE 1

The following compositions were prepared (in g of starting materials).

The thready effect was evaluated when a value scale of 0 to 10 with the following evaluation

10=thready effect very present and

0=no thready effect.

The thready effect was evaluated after 1 to several weeks of storage of the composition of 45° C.

### Protocol for Evaluating the Thready Effect

For each composition, a small amount of milk have been poured into the palm of the hand. The composition was then massaged with the other palm of of the hand (2-3 massages), then the two palms of the hands have been separated and the thready/fibrous effect of the composition after massaging was measured. The presence or absence of fibers between the two palms have been assessed. It can be seen that only the compositions of the invention made it possible to retain the texturizing fiber effect after 5 weeks of storage at 45° C.

### EXAMPLE 2

The following compositions were prepared (in g of starting materials).

TABLE 2

| | A | |
|---|---|---|
| INCI | invention | B |
| CITRIC ACID | 0.03 | 0.03 |
| PHENOXYETHANOL | 0.7 | 0.7 |
| CETEARYL ALCOHOL | 3 | 3 |
| Mixture of myristyl stearate and | 0.5 | 0.5 |
| myristyl palmitate | | |
| ALOE BARBADENSIS LEAF JUICE | 0.1 | 0.1 |
| FRAGRANCE | 0.5 | 0.5 |
| PEG-90M | 0.5 | 0.5 |
| PULLULAN | 4 | 0.5 |
| AMODIMETHICONE (and) | 0.8 | 0.8 |
| TRIDECETH-6 (and) | | |
| CETRIMONIUM CHLORIDE | | |
| BUTYLENE GLYCOL | 2 | 2 |
| POLYSORBATE 20 | 0.8 | 0.8 |
| BEHENTRIMONIUM CHLORIDE | 0.8 MA | 0.8 |
| WATER | qs 100 | qs 100 |

Each of the compositions A and B were applied, in a proportion of 0.5 g of composition per lock, to locks of straight natural caucasian hair of 2.7 g, 27 cm long, prewashed with a shampoo (DOP), rinsed and predried using a towel. The locks thus treated were rolled around a spiral roller and left to dry for 24 hours at ambient temperature.

The rollers were then removed.

For each lock, the appearance and the fixing, that is to say whether the hair was set (a great deal of fixing), were evaluated.

Each lock was suspended vertically in front of graph paper and were then placed in a chamber at controlled humidity (80% RH).

The locks obtained were then photographed after 24 hours and the length of each lock was measured.

| | A | B |
|---|---|---|
| | (invention) | (comparative) |
| Length after 24 h at 80% | 22.8 cm | 24.5 cm |
| relative humidity | | |
| % hold after 24 h | 15% | 9.2% |

It was noted that the composition according to the invention using composition A made it possible to improve the hold of the hairstyle after 24 hours compared to composition B comprising 0.5% of polysaccharide. Further the curls were better defined with composition A than with composition B.

1.-18. (canceled)

19. A composition for treating keratin fibers, comprising:
at least one fatty substance,
at least one polysaccharide, present in an amount greater than or equal to 3.0% by weight, relative to the total weight of the composition,
at least one diol having a molecular weight of less than 1000, and
at least one polyethylene glycol comprising from 10,000 to 200,000 oxyethylene units.

20. The composition according to claim 19, wherein the fatty substance comprises at least one fatty alcohol, at least one fatty ester, or mixtures thereof.

21. The composition according to claim 19, wherein at least one fatty substance is solid at ambient temperature.

22. The composition according to claim 19, wherein the at least one fatty substance comprises a mixture of a C₁₆-C₁₈ fatty alcohol and of fatty esters obtained from myristyl alcohol.

23. The composition according to claim 19, wherein the at least one diol comprises a diol having a molecular weight of less than 500.

24. The composition according to claim 19, wherein the at least one diol comprises a diol having a molecular weight of less than 100.

25. The composition according to claim 19, wherein the polyethylene glycol comprises from 50,000 to 100,000 oxyethylene units.

26. The composition according to claim 19, wherein the polyethylene glycol comprises approximately 90,000 oxyethylene units.

27. The composition according to claim 19, wherein at least one of the polysaccharides is chosen from microbial gums.

28. The composition according to claim 19, comprising a scleroglucan gum and/or pullulan.

29. The composition according to claim 19, which is in emulsion form.

30. The composition according to claim 19, comprising at least 50% by weight of water, relative to the total weight of the composition.

31. The composition according to claim 19, having a viscosity of less than 5000 mPa·s.

32. The composition according to claim 19, comprising an amount of fatty substance ranging from 0.01% to 20% by weight, relative to the total weight of the composition.

33. The composition according to claim 19, comprising an amount of polysaccharide ranging from 3.0% to 20% by weight, weight relative to the total weight of the composition.

34. The composition according to claim 19, comprising an amount of diols ranging from 0.1% to 25% by weight, relative to the total weight of the composition.

35. The composition according to claim 19, comprising a quantity of polyethylene glycol ranging from 0.01% to 20% by weight, relative to the total weight of the composition.

36. A composition for treating keratin fibers, comprising:
at least one fatty substance chosen from fatty alcohols, fatty esters, or mixtures thereof, wherein the at least one fatty substance is predominantly solid at ambient temperature,
at least one polysaccharide chosen from scleroglucan gum, pullulan, or mixtures thereof, present in an amount greater than or equal to 3.0% by weight, relative to the total weight of the composition,
at least one diol having a molecular weight of less than 100, and
at least one polyethylene glycol comprising from 50,000 to 100,000 oxyethylene units.

37. A process for shaping and/or conditioning keratin fibers, comprising applying to said keratin fibers a composition comprising:
at least one fatty substance,
at least one polysaccharide, present in an amount greater than or equal to 3.0% by weight, relative to the total weight of the composition,
at least one diol having a molecular weight of less than 1000, and
at least one polyethylene glycol comprising from 10,000 to 200,000 oxyethylene units.

38. The process according to claim 36, for defining the hair curls and/or styling the head of hair.
